(19) United States
(12) Patent Application Publication
(10) Pub. No.: US 2006/0178675 A1
(43) Pub. Date: Aug. 10, 2006

(54) System, device, and method for providing access in a cardiovascular environment

(75) Inventor: Baron L. Hamman, Dallas, TX (US)

Correspondence Address: BAKER BOTTS L.L.P., 2001 ROSS AVENUE, SUITE 600, DALLAS, TX, 75201-2980, US

(73) Assignee: Castlewood Medical Technologies LLC

(21) Appl. No.: 11/347,542

(22) Filed: Feb. 3, 2006

Related U.S. Application Data
(60) Provisional application No. 60/651,690, filed on Feb. 10, 2005.

(51) Int. Cl. A61F 11/00

(52) U.S. Cl. 606/108

(57) ABSTRACT

A device to be used in a cardiovascular environment, comprising an expandable element that is coupled to a rod and that includes a compressed state and an expanded state. The expandable element is operable to be positioned within a wall of an organ while the expandable element is in the compressed state. The expandable element is further operable to be deployed once it is within the organ such that it is in the expanded state. The device further includes a cutter element operable to make a circular incision at the wall of the organ. The expandable element creates a resistive force when it is pulled against the wall while in the expanded state such that an interface is created for making the incision. The device includes a stop-grip mechanism that maintains the resistive force at the interface. One embodiment features the expandable element being umbrella shaped.

### RELATED APPLICATIONS

This Application claims the benefit under 35 U.S.C. § 119(e) of U.S. Provisional Application Ser. No. 60/651,690 entitled: “Devices for Accessing Interior Chamber of the Heart and Device for Aiding Construction of Valved Conduits from the Heart,” which was filed on Feb. 10, 2005, and which is incorporated by reference herein.

### TECHNICAL FIELD OF THE INVENTION

This invention relates in general to the field of cardiac and vascular surgery and, more particularly, to a system, a device, and a method for providing for access in a cardiovascular environment.

### BACKGROUND OF THE INVENTION

The treatment of vascular diseases has grown exponentially in terms of sophistication and diversity. One area of interest relates to the ability to access the inside of the heart in order to touch, cut, move, paint, or burn areas of the heart in order to change its function, shape, conduction pattern, or to ablate a normal or an abnormal rhythm pattern.

Another area of interest pertains to the treatment of deficiencies in the heart and its chambers, valves, and vessels emanating therefrom. In certain cases of aortic stenosis or left ventricular outflow tract obstruction, surgeons have provided relief to patients by implanting a prosthetic valved conduit: extending from the apex of the left ventricle to the aorta. This conduit provides an outflow tract for flow exiting the left ventricle. The surgery leaves the natural outflow tract intact and untouched. This surgical technique has proven useful in cases of congenital or acquired supravalvular, valvular, and subvalvular stenoses where more conventional approaches (such as aortic valvotomy or commissurotomy) produce inferior results due to the severity of the obstruction. These substandard results may also be attributable to difficulties in affecting an accurate obstruction relief, or due to dropping debris from the attended valve (or other similar component). The debris can readily create an embolus that is free to travel with the blood blow and, potentially, cause a stroke (in the case of lodging in the brain) or other bodily injuries.

In more recent years, prosthetic conduits with valves have enjoyed substantial notoriety. Their popularity is due to their tremendous success rate, their efficacy, and their ability to offer extraordinary benefits to a patient.

Note that such cardiac procedures pose certain problems for a surgeon. For example, a surgeon is generally confined or restricted in his movements during the surgery, which may be due, in part, to instrumental limitations. A surgeon must often complete a number of sophisticated tasks during a given procedure. Some of these tasks should be completed somewhat concurrently or even simultaneously. Therefore, optimizing or simplifying any of these steps may yield a significant reduction in burden for a surgeon. Additionally, with the elimination of perfunctory tasks and tedious chores, the surgeon is then free to shift his attention where it is most needed: on the procedure itself.

Moreover, many surgical instruments that address issues at the apex of the heart are cumbersome, difficult to manipulate, potentially harmful to patients, and clumsy or awkward in many situations. Their deficiencies create a significant challenge for the surgeon, who is already being taxed by a number of arduous tasks. In addition, many current devices are unacceptable because they cause trauma and inflammation issues for the patient or because they have a propensity to cause strokes.

Accordingly, the ability to provide an effective medical instrument that properly accounts for the aforementioned problems presents a significant challenge for component manufactures, system designers, and surgeons alike.

### SUMMARY OF THE INVENTION

From the foregoing, it may be appreciated by those skilled in the art that a need has arisen for an improved instrument for achieving superior control, management, and performance during a procedure that offers optimal access at a targeted surgical site. In accordance with an embodiment of the present invention, a device, a system, and a method for enhancing an operation involving access (particularly, but not limited to, left ventricular access) are provided that includes a flexible, highly precise, easy-to-use device, which substantially eliminates or greatly reduces disadvantages and problems associated with conventional equipment and instruments.

A device to be used in a cardiovascular environment, comprising an expandable element that is coupled to a rod and that includes a compressed state and an expanded state. The expandable element is operable to be positioned within a wall of an organ while the expandable element is in the compressed state. The expandable element is further operable to be deployed once it is within the organ such that it is in the expanded state. The device further includes a cutter element operable to make a circular incision at the wall of the organ (while sealing the intraorgan fluids inside). The expandable element creates a resistive force when it is pulled against the wall while in the expanded state such that an interface is created for making the incision. The device includes a stop-grip mechanism that maintains the resistive force at the interface (to allow unhanding of the device).

In a particular embodiment, the device is of a size sufficient to allow a sealed access to a cavity of the organ for placement, manipulation, or repair of a heart valve. A handle (e.g. an angulated rod handle) can be used to manipulate (and potentially lock) the expandable element into its compressed state and the expanded state.

Certain embodiments of the present invention may provide a number of technical advantages. For example, the present system can include a simple locking mechanism for maintaining a position of the device. The locking mechanism allows a surgeon to unhand the device and, thereby, offers exceptional flexibility and adaptability for a surgeon. Moreover, the operation of the device allows the port to be “dropped” back into the chest (i.e. not directly handled by the operator), while another device or tool is being readied for use. Still other advantages of the device would include minimizing blood loss during intra-ventricular surgical repair, while maintaining a pathway to minimize time during tool changes. The device allows for a number of beating heart surgeries to occur and, further, avoids the complications associated with heart-lung machines.

In addition, the present invention offers increased accuracy for a surgeon, who is relegated the difficult task of making a precise circular incision in the wall of the heart. Additionally, the device can access and seal a variety of ventricular or cardiac tissue walls.

Also, the proposed platform would allow a surgeon to insert a valve (or some other implantable device) through the interior of the port although it is not actually part of the implantable device. Additional details relating to these advantages are described below with reference to corresponding FIGURES.

Certain embodiments of the present invention may enjoy some, all, or none of these advantages. Other technical advantages may be readily apparent to one skilled in the art from the following figures, description, and claims.

### BRIEF DESCRIPTION OF THE DRAWINGS

To provide a more complete understanding of the present invention and features and advantages thereof, reference is made to the following description, taken in conjunction with the accompanying figures, wherein like reference numerals represent like parts, in which:

FIGS. 1A-1C are simplified schematic diagrams that illustrate a top view of a device to be used in a surgical procedure in accordance with one embodiment of the present invention;

FIG. 2 is a simplified schematic diagram of a heart and a valve conduit, which are representative of a scenario in which the device of FIG. 1 may be applicable;

FIGS. 3A-3B are simplified schematic diagrams of a top view of the device in which a cutter element is provided thereon;

FIGS. 4A-4C are simplified schematic diagrams of a top view of the device in combination with a tube that facilitates various operations of the device; and

FIGS. 5A-5G are simplified schematic diagrams of example configurations of the device in which various portions of the device have been removed.

### DETAILED DESCRIPTION OF THE INVENTION

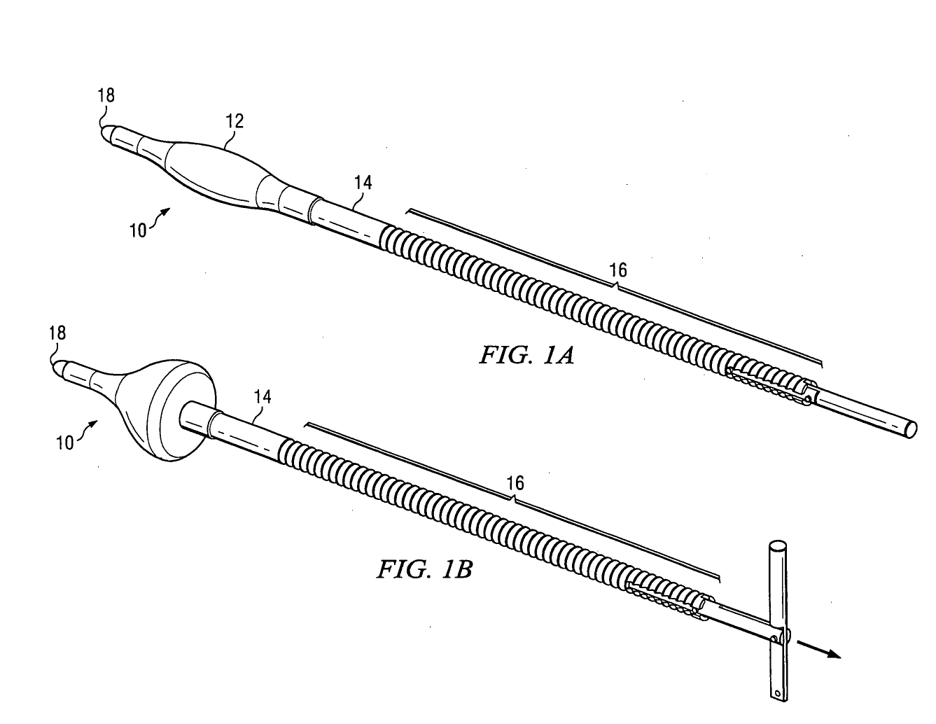

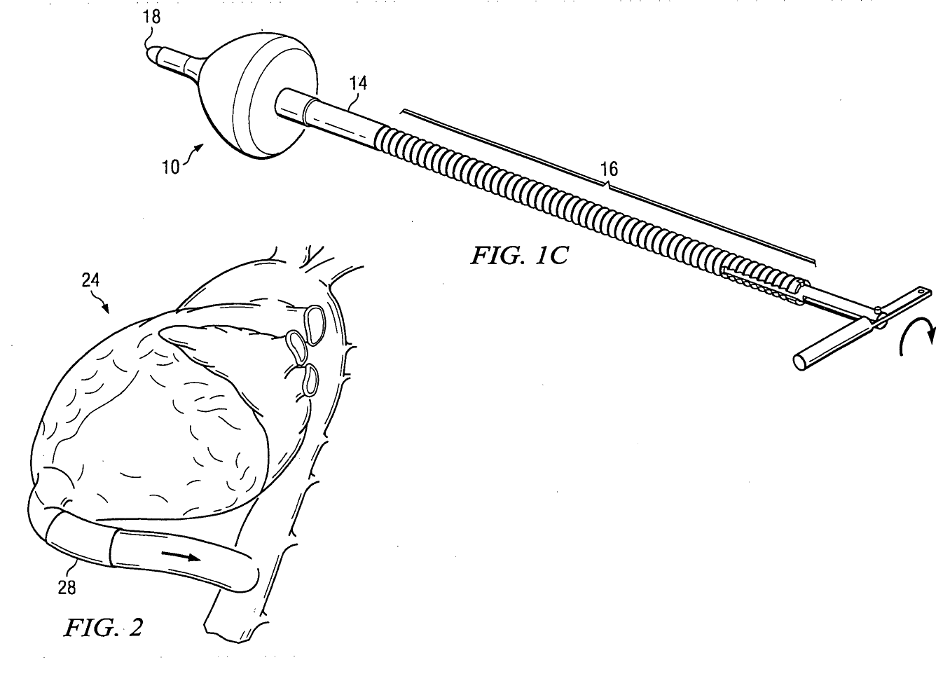

FIGS. 1A, 1B, and 1C are simplified schematic diagrams that illustrate a device 10 for creating an effective point of entry at a targeted location. In one embodiment, the targeted location is the apex of the heart. However, the targeted location can be any other suitable location in the body in which a small piece of tissue is sought to be removed by a surgeon or where a sealed access to a fluid or gas containing organ is desired. Device 10 includes an expandable element 12 (which is umbrella shaped in one embodiment) and a rod 14, which includes a threaded portion 16. Device 10 also includes an obturator 18, which is olive shaped and which facilitates a smooth streamlined entry for the attending surgeon. In one embodiment, obturator 18 is blunt at its end such that it does not pierce a valve (or other delicate structure) that may be subsequently placed, removed, burned, ablated, or manipulated by the surgeon. Obturator 18 does have enough sharpness or rigidity such that it accomplishes some directionality. In an alternative embodiment of the present invention, obturator 18 may be replaced by any other suitable element that facilitates an efficient entry at the targeted location for the surgeon.

Additionally, obturator 18 may be coupled to an accompanying handle for purposes of actuation, as is illustrated in FIG. 1C. Thus, an angulated rod handle driver, with an accompanying pin structure, can be provided to device 10. A sleeved locking mechanism may be used to manipulate expandable element 12 to either its compressed or its expanded state. Specifically, FIG. 1A illustrates the handle in an inactive mode such that expandable element 12 is in a collapsed state, whereas FIG. 1B illustrates device 10 where the handle is being employed. FIG. 1C illustrates the handle being used to deploy expandable element 12 such that the umbrella is fully expanded.

Expandable element 12 and rod 14 are constructed of a hard plastic material in one embodiment. In other embodiments, expandable element 12 and rod 14 are constructed of any suitable polymer or composite material. These elements may be coupled to each other in any appropriate fashion, or these items may simply be integral. Alternatively, the design of device 10 may be changed, enlarged, or modified considerably in order to accommodate particular arrangements or configurations. The illustrated examples of FIGS. 1A and 1B can be altered considerably without departing from the broad teachings of the present invention.

Expandable element 12 is larger than the cutter by about 2-8 mm in diameter (or greater or less, depending upon particular applications) to allow for stability of the system after a cutter (which is described in greater detail below) removes a concentric circular piece of tissue. In still another embodiment, expandable element 12 has a diameter of approximately 18-30 mm in the expanded state (FIG. 1A) and 8-12 mm in diameter in the initial collapsed configuration (FIG. 1B).

In alternative embodiments, device 10 can serve as a chassis for mounting a valved conduit. The valved conduit system could allow therapy for patients who are not candidates for traditional therapies such as cardio pulmonary bypass, or for patients having aortic calcification or calcified stenosis of the valves.

The access port system can also be used in conjunction with any other system to provide access to the ventricle, to the atrium, or to any large intra-cardiac or intravascular structure while the heart is still beating and may or may not be pumping blood. In specific applications, device 10 could be used as an access port for mitral or aortic valve manipulation, replacement, repair, or for atrial fibrillation procedures or ventricular wall geometric procedures.

Note that, currently, there are few relevant devices available on the market for solving a number of problems associated with beating heart left ventricular (or intracardiac) access. Generally, interventions require surgical skill to perform placement of the valved conduits. Access to the ventricle has previously been established by cutting a linear incision and by performing a dilation of the linear incision in order to gain access. The linear incision is then subsequently repaired by sutures. Device 10 offers an improved design and provides an instrument capable of readily accessing the interior chamber of the heart: providing a hemostatic sealed port for introducing tools into the interior chamber of the heart.

Before proceeding further, for purposes of teaching and discussion, it is useful to provide some overview as to the way in which the following invention operates. The following foundational information may be viewed as a basis from which the present invention may be properly explained. Such information is offered earnestly for purposes of explanation only and, accordingly, should not be construed in any way to limit the broad scope of the present invention and its potential applications.

An example environment in which device 10 can operate is illustrated by FIG. 2. FIG. 2 is a simplified schematic diagram that illustrates a heart 24, which includes a valve conduit system 28 connected to the descending aorta ‘y.’ An arrow is illustrated as showing blood flow from the apex of heart 24 to the aorta ‘y.’ Many existing systems attempt to achieve relief of an obstruction to the outflow of the left ventricle by opening or replacing the aortic valve. The aortic valve location has previously been a popular choice because it is a somewhat ‘natural’ solution to employ. However, incising a hole in the apex of the heart (i.e. at the bottom of the heart) is also a viable solution. Thus, instead of pumping out of the aortic valve, which may be diseased, narrowed, or somewhat occluded, a suitable outflow can be achieved at the bottom of the heart through a hole and, subsequently, an implantable valve that facilitates one-way blood flow can be used. Hence, the apex of the heart could readily be used for placement of a valve conduit. However, such a procedure is not without its flaws, for example this paradigm is generally considered to be cluttered and somewhat messy.

Aside from the aforementioned procedural flaws, there are also a number of additional issues that a surgeon should be aware of in attempting to incise a hole at the apex of the heart. For example, one problematic issue in such an environment involves the use of a cardiopulmonary bypass pump. Another problematic issue relates to clamping of the aorta. Inherent in both of these issues is the potential for strokes.

The present invention solves these issues, and others, by providing a suitable protective barrier, which would keep much of this clutter out of the surgeon's way. Specifically, the present invention can utilize an umbrella-shaped design, whereby device 10 can be easily inserted into (and removed from) the target location in a collapsed state. In a compressed or collapsed state, device 10 is generally small in relation to the incised hole. Once suitably positioned, device 10 can be deployed, where it is free to expand. By pulling back on device 10, the umbrella design can expand to an area greater than the incised hole. Hence, the surgeon can stabilize the environment by simply creating enough pressure between device 10 and the wall of the heart.

Such an arrangement is ideal, as it offers tremendous freedom and maneuverability for the surgeon. With device 10 suitably positioned, the surgeon is free to sew in a valve conduit, or any other device, at this surgical location. Once the valve conduit (or any other element) is sewn, then the umbrella portion of device 10 can be undeployed (i.e. collapsed) and removed from the site.

One advantage provided by device 10 is that it is capable of making a circular cut that approximates a valve conduit (or any other element sought to be placed at the surgical site). A circular cut is important for achieving a superior seal. Device 10 also augments flexibility and convenience for a surgeon because it is capable of locking into place once it is suitably positioned.

Another important advantage offered by device 10 is that it affords the surgeon the ability to always have a “completed procedure.” For example, if for some reason the objectives of the surgeon are not being met during the initial surgery, he can simply shift his strategy, place a valve conduit at the surgical site, and necessarily resolve this patient in some fashion: even if resolution involves a strategy that was different from the originally intended objective. This is in contrast to other, more limited systems and devices that offer an all-or-nothing proposition. Consider the case of a simple stent procedure that, for some arbitrary reason, goes awry. While in a catheter laboratory, the surgeon cannot avail himself to alternative surgical options. Hence, these stent procedures are not amenable to any shift in operational strategy. However, device 10 is adaptive, as some of its components can easily be manipulated to achieve objectives that are different from those originally proposed.

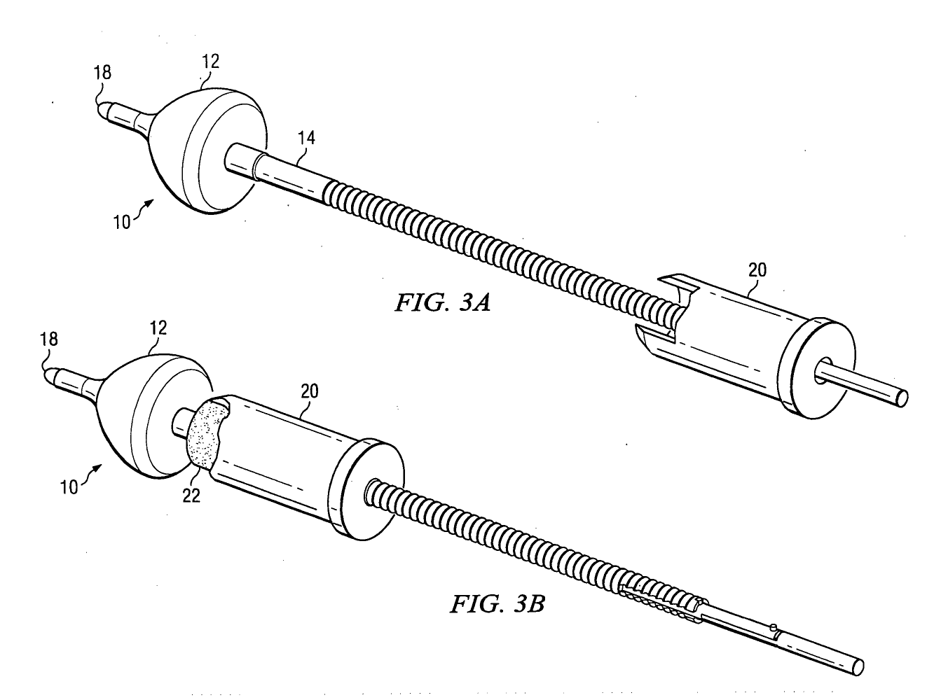

FIGS. 3A and 3B are simplified schematic diagrams that further illustrate a cutter element 20 of device 10. Cutter element 20 has a certain sharpness such that it is operable to incise a hole that is smaller than expandable element 12. Cutter element 20 is somewhat hollow or concave such that it can incise a hole and retain the incised portion of tissue. The incised portion would be of a doughnut shape and, further, could be used for the purpose of biopsy where appropriate. Once the tissue has been effectively removed, then the surgeon could position a valve conduit (e.g. with self-attaching legs) in the hole and secure the valve. Once the valve is properly secured, then expandable element 12 is undeployed or collapsed and subsequently removed.

Cutter element 20 is secured to rod 14 through threaded portion 16 such that rotation of cutter element 20 causes it to move toward expandable element 12. In other embodiments, cutter element 20 may move along rod 14 in any suitable fashion (e.g. spring loaded mechanisms, ratcheting configurations, notching arrangements where rod 14 is designed to include specific locations at which cutter element 20 can be secured, simple cam configurations, etc.).

Device 10 can leverage expandable element 12 (or any other collapsible item that can provide the requisite seal) to provide for stabilization and, further, to produce a hemostatic effect on the interior of the heart. For an introduction at the surgical site, expandable element 12 of device 10 is in its collapsed state with the deployable member at its minimum profile over the shaft (e.g. 10 to 14 mm diameter).

The inner shaft of device 10 initially has a dilator positioned within the inner diameter and which extends out from the distal tip of device 10. The surgeon can make a small cut in the wall of the heart at the desired location for the access port. Device 10 is then inserted through the wall of the heart and when expandable element 12 is fully within the heart, the outer shaft of device 10 is manipulated relative to the inner shaft to deploy the expandable member. The dilator can be removed so that access through the wall is permitted.

As highlighted above, expandable element 12 can be a braid, a balloon, a malecot, or any other suitable component that is capable of being collapsed from its original state to a smaller state for introduction into the targeted site. Once suitably positioned or installed, expandable element 12 can then be deployed such that it occupies a larger diameter. This maintains its position and provides a natural hemostasis.

For example, a braid (that serves as expandable element 12) can be used in such an arrangement, which can be shaped to provide an optimal sealing against the interior heart chamber. The braid could be coated with an elastomeric membrane (e.g. such as silicone or polyurethane) to provide a sealed surface. Device 10 may be made with a dense enough braid or fiber structure to allow the fibers alone to provide suitable resistance to the passage of blood.

With device 10 appropriately positioned and with the expandable member deployed, a small amount of traction can be applied to the shaft in order to seat the expandable member against the interior wall of the heart. At this point, the surgeon can perform a simple thumb manipulation (e.g. a thumb wheel) to advance the exterior foot against the exterior surface of the heart. A simple spring can be provided to take up some amount of deflection of the wall thickness due to contraction of the heart muscle and, further, to provide a measure of safety against over compressing the heart muscle and, thereby, causing pressure necrosis. The thumb wheel can be advanced until the spring is partially or fully compressed; visual monitoring will allow the surgeon to see when the spring is fully compressed such that advancement is no longer required. From this point, the access port is installed and ready to use.

At the proximal end of device 10, a hemostatic valve can be used to allow devices to be placed and removed while minimizing blood loss. The valve uses two separate features for sealing. With no tool in place, a modified duckbill valve (or any other suitable valve system) can be used to provide the hemostatic effect. With a tool placed through the valve, a circumferential wiper seal can be used to allow the tool to be moved within the seal and to provide for a range of tool sizes to be accommodated.

To remove device 10, the thumb wheel (or any other suitable releasing mechanism) can be rotated to release tension of the foot against the outer heart surface. The inner and outer shafts can then be manipulated, relative to one another, to collapse expandable element 12. Device 10 can then be gently withdrawn from the wall of the heart. After device 10 is removed, the initial cut can be sutured to close the hole and, further, the hemostasis can then be verified.

In operation of yet another example procedure, device 10 is generally pulled to seat the expandable member against the inner surface of the heart. At this point, cutter element 20 is installed onto the shaft and advanced down to the heart wall. Cutter element 20 may ride on any suitable advancing mechanism such as a thread or a cam (or any other suitable advancing mechanism), which advances the cutter. Additionally cutter element 20 has a distal sharpened end, which cuts tissue as it is rotated or advanced into the tissue. Cutter element 20 also includes a tissue retaining feature such as an interior ridge or internal barbed spikes that retain the cut tissue in the cutter. In this one non-limiting example embodiment, cutter element 20 is in a range of 13 to 25 mm in diameter.

After cutting and removing the circular tissue, continued traction on the expanded braid maintains hemostasis of the hole. A heart valve and vascular conduit can then be installed over the shaft of the device 10 and positioned into the cut hole in the heart. This conduit can be attached to the heart via suturing or other suitable methods where appropriate. With the valve and conduit installed and sutured to the heart, the expandable member can be released and hemostasis of the valve and heart attachment verified. Once the valve placement is verified, and the surrounding area is sutured and sealed, the expandable member can be collapsed and then gently removed from within the valve and conduit. Finally the conduit can be attached to the aorta or other suitable structure and the final anastomosis completed.

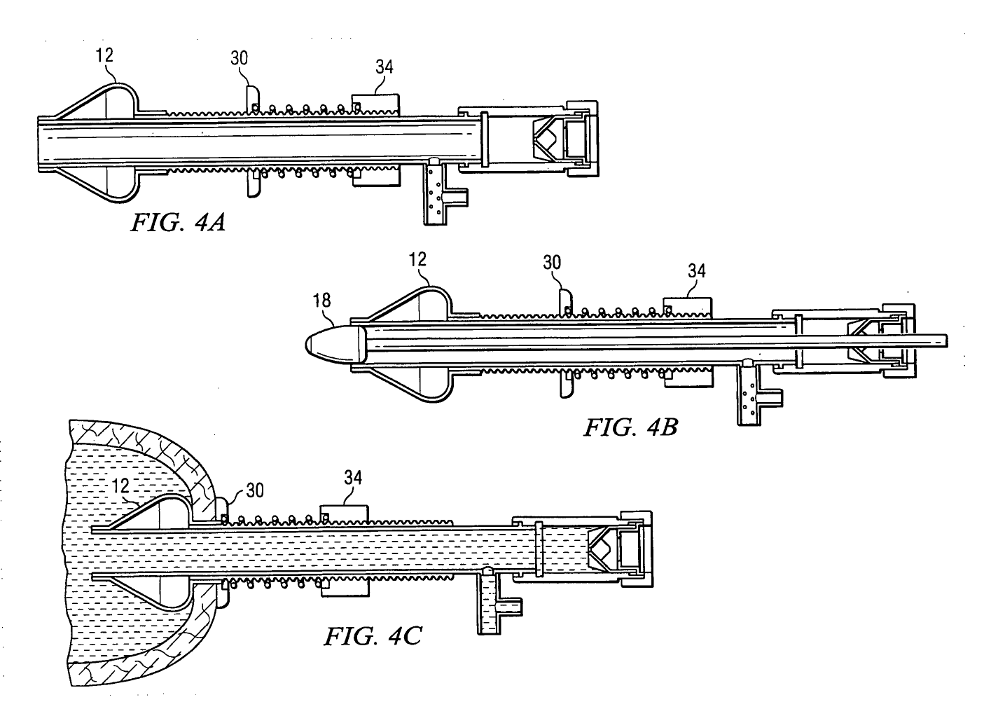

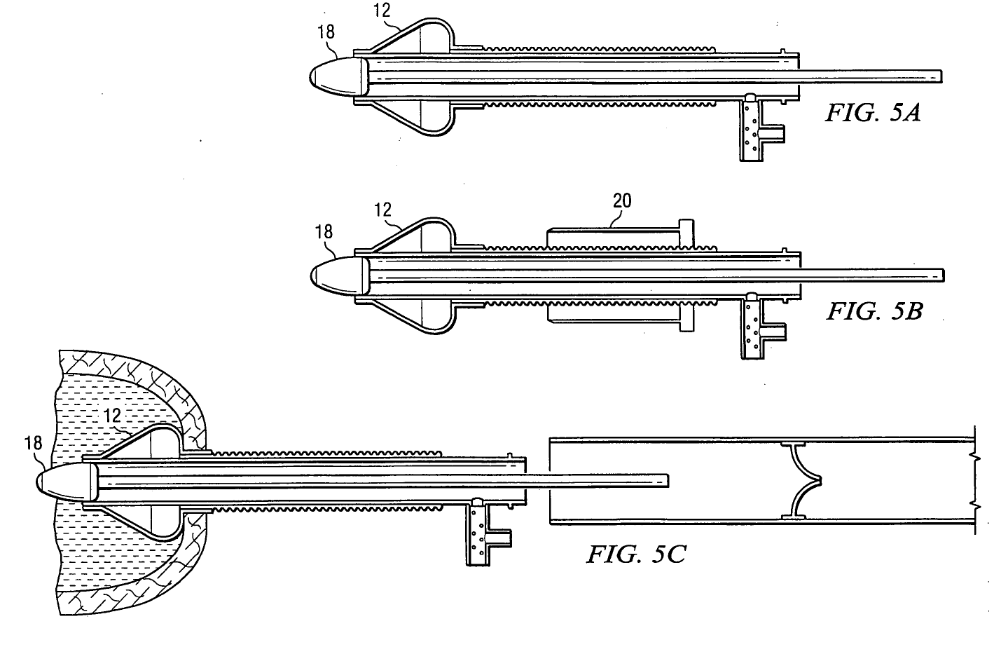

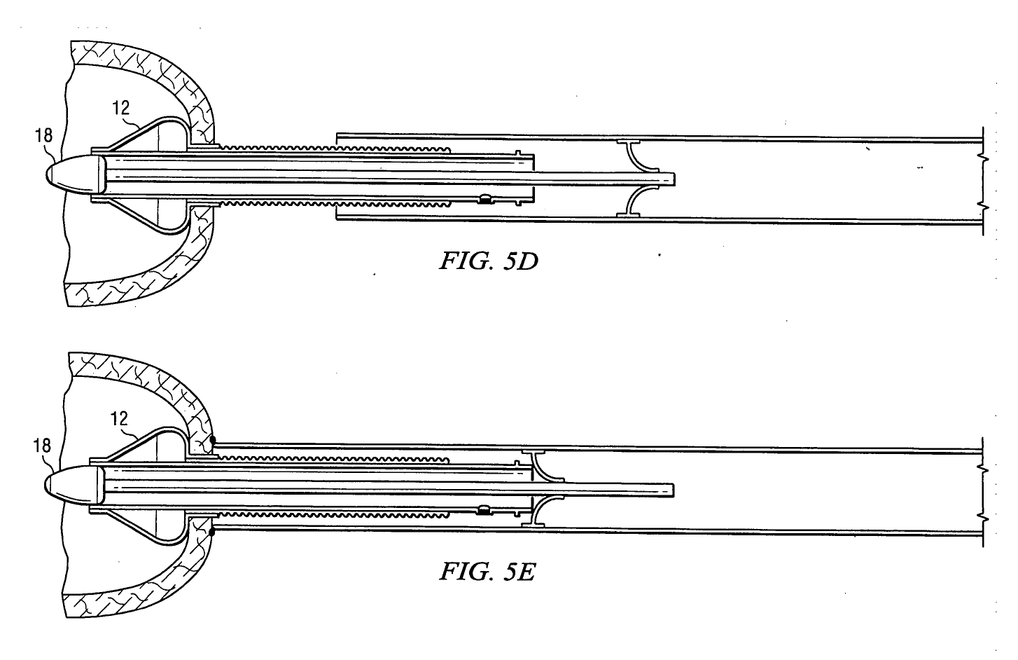

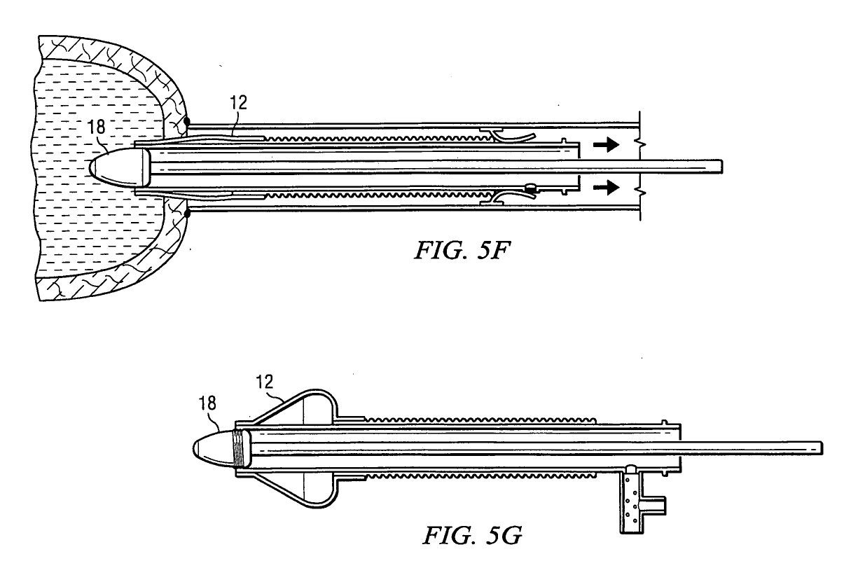

FIGS. 4A-4C are simplified schematic diagrams illustrating a top view of device 10. As an overview, it should be understood that FIGS. 4A-5C illustrate significant modifications in scale and in configurations, as compared to the embodiments of FIGS. 1A-1C and 3A-3B. These new design choices are based on previous experimental work and, at least in one instance, particular system needs. For example, the width of device 10 in FIG. 4A (at its widest point) is approximately 30-35 millimeters. The embodiment of FIG. 4A includes a long tube that can enter and exit the cavity of the heart easily without creating unnecessary leakage. Again, based on one set of particular design choices, the width of the tube is about 0.5 millimeters. It is critical to note that these measurements are only offered as examples, as any permutations or alternations in these specifications are clearly within the broad scope of the present invention. Accordingly, these measurements should not be construed to limit the present invention in any way.

As is illustrated in FIGS. 4A and 4B, device 10 also includes a spring mechanism that is facilitated by a stop 34 (which serves as an anchoring element) and a grip element 30. Together, these two elements form a ‘stop-grip mechanism’ that operates to hold device 10 in one position. [As used herein in this document, the term ‘stop-grip mechanism’ connotes any component (e.g. spring loaded, friction-based systems, ratcheting configurations, etc.) that offers the ability to secure device 10 into a designated position. This feature allows increased freedom for the surgeon, who has been delegated to perform the surgery, as explained repeatedly herein.)

Specifically, applying pressure to these two components (i.e. a squeezing force) causes a compression and then a subsequent resistive force, which secures device 10 in a specified position. This locking feature affords the aforementioned liberation to a surgeon who, while device 10 is stable, is able to perform other tasks while device 10 remains in its seated position. Thus, the present invention offers increased flexibility to the surgeon because it can seat and remain in its intended position, while the surgeon utilizes other medical instruments or performs other tasks. The present invention employs a spring mechanism that eliminates the need for a surgeon to constantly apply pressure in holding the device in a fixed position. Device 10 allows for an easy entry and exit from the tube via a diaphragm, a plastic valve, or any other element that facilitates such movement.

Device 10 also includes a sump element 40, which operates to release or exhaust air bubbles or other debris that is present in the system. Sump element 40 is removable. At one end, which generically represents the handle end of the instrument, device 10 includes a simple valve that offers the ability to receive objects at device 10. In one embodiment, the valve is a window valve that is less cumbersome than conventional valves. However, device 10 could readily employ conventional valves, or any other type of conduit that could easily facilitate the teachings of the present invention, as outlined herein.

In operation of another example embodiment used to illustrate some of the applications of the present invention, consider the case of a patient who is experiencing some pain that emanates from the left side of their heart or the pumping chamber generally. The pathology of the patient could be simple stenosis or it could involve atrial fibrillation, a cardiac tumor, etc. In other instances, the patient could require a change in the shape of the ventricle itself by remodeling, sewing, placating, or by placing a patch in a targeted area. All of these issues require the surgeon to have access to the inside of the ventricle.

As an initial step, the surgeon may use device 10 in creating a small hole in the apex of the heart. Expandable element 12 occupies part of the ventricle because of its shape and because the tissue is somewhat malleable. Hence, when a somewhat rigid expandable element 12 is pulled against this tissue, a viable seal is created. Once the seal has been created, other components of device 10 can be utilized on the other side of expandable element 12 to essentially squeeze the wall and to lock device 10 into a specific position. Now, the access device can be dropped or unhanded such that the surgeon can focus on other tasks at hand without having to hold device 10. This shift in emphasis, from focusing on maintaining a fixed position of an instrument to concentrating on the procedure itself, is critical to the success of any medical procedure. By employing device 10, the surgeon is no longer burdened with menial or tedious chores; instead, his attention is on the surgery itself. Note that device 10 allows access to the intracardiac chamber without entraining air and without dropping removed portions of tissue. Further, device 10 allows the surgeon to use other instruments while in the chamber (e.g. forceps, a laser, a scope or other visualization instrument, etc.).

FIG. 4C illustrates how the bracing holder of device 10 is moved laterally toward the distal end of the instrument. Grip 30 is now interfacing with the ventricular wall, whereby blood is properly sealed. In addition, obturator is removed in the embodiment of FIG. 4C.

FIGS. 5A-G are simplified schematic diagrams of a top view of device 10. FIG. 5A simply illustrates device 10 without the valve, while FIG. 5B illustrates device 10 with cutter element 20 positioned over the tube. Based one set of particular design choices, the width of cutter element 20 is about 18 millimeters and the inner rod 14 is about 15 millimeters. Once again, the audience should be reminded that these measurements are only offered as examples, as any permutations or alternations in these specifications are clearly within the broad scope of the present invention.

FIG. 5C is a simplified schematic diagram of a top view of device 10 after a number of components have been removed. The removal of these components could be the result of a shift in objectives for the attending surgeon. As highlighted above, the surgeon is afforded the capability of abandoning the originally intended procedure in certain situations. For example and with reference to FIG. 5C, after a surgeon recognizes that he must change his strategy, the surgeon could reinsert the obturator, remove the diaphragm, remove the diaphragm housing, take off sump 40, and discard stop 34 and gripping element 30. The surgeon could then position cutter element 20 over the apparatus to allow for a circular cut to be made. In this instance, the cut will allow for a valve conduit to be suitably positioned at the surgical site. The valve conduit can be slipped over the outside of the apparatus and then fixed to the wall of the heart. Then, expandable element 12 can then be retracted and removed from the valve conduit itself.

Thus, as is illustrated, FIG. 5D shows the removal of the stop cock. FIG. 5E illustrates a number of sutures that are made at the ventricular wall. The sutures are illustrated as small notches in this depiction. Furthermore, expandable element 12 can be shaped as a concave umbrella such that a surgeon has ample room to perform appropriate suturing. In a sense, expandable element 12 could be provided with a lip that facilitates this operation. FIG. 5F illustrates expandable element 12 in its collapsed state such that it can be removed from the surgical site.

FIG. 5G illustrates an alternative embodiment of the present invention. In this embodiment, obturator 18 is fitted with threads such that it can be removed from, or secured to, an inner sheath to create an appropriate seal. In such a scenario, there are two tubes (or sheaths) present that allow for a number of components to be removed without breaking the seal.

Note that any of the previously discussed materials could be included in a given kit, which could ostensibly be provided to a surgeon who is responsible for performing a cardiovascular procedure. A basic kit could include device 10, along with an accompanying tube and a valve to be used in conjunction with device 10. Any of these components may be manufactured based on particular specifications or specific patient needs. The present invention contemplates considerable flexibility in such components, as any permutation or modification to any of these elements is clearly within the broad scope of the present invention.

It is important to note that the stages and steps in the preceding FIGURES illustrate only some of the possible scenarios that may be executed by, or within, the architecture of the present invention. Some of these stages and/or steps may be deleted or removed where appropriate, or these stages and/or steps may be modified or changed considerably without departing from the scope of the present invention. In addition, a number of these operations have been described as being executed concurrently with, or in parallel to, one or more additional operations. However, the timing of these operations may be altered considerably. The preceding example flows have been offered for purposes of teaching and discussion. Substantial flexibility is provided by the proffered invention in that any suitable arrangements, chronologies, configurations, and timing mechanisms may be provided without departing from the broad scope of the present invention.

Note also that the example embodiments described above can be replaced with a number of potential alternatives where appropriate. The processes and configurations discussed herein only offer some of the numerous potential applications of the device of the present invention. The elements and operations listed in FIGS. 1A-5C may be achieved with use of the present invention in any number of contexts and applications. Accordingly, suitable infrastructure may be included within device 10 (or cooperate with device 10) to effectuate the tasks and operations of the elements and activities associated with managing a bypass procedure.

Although the present invention has been described in detail with reference to particular embodiments in FIGS. 1A-5C, it should be understood that various other changes, substitutions, and alterations may be made hereto without departing from the sphere and scope of the present invention. For example, although the preceding FIGURES have referenced a number of components as participating in the numerous outlined procedures, any suitable equipment or relevant tools may be readily substituted for such elements and, similarly, benefit from the teachings of the present invention. These may be identified on a case-by-case basis, whereby a certain patient may present a health risk factor while another (with the same condition) may not. Hence, the present device may be designed based on particular needs with particular scenarios envisioned.

It is also imperative to note that although the present invention has been illustrated as implicating a procedure related to the apex of the heart, this has only been done for purposes of example. The present invention could readily be used in any cardiovascular procedure and, accordingly, should be construed as such. For example, the present invention can be used in applications involving the stomach, bladder, colon, bowels, etc. The present invention may easily be used to provide a viable vascular management solution at various locations of the mammalian anatomy, which are not necessarily illustrated by the preceding FIGURES.

Numerous other changes, substitutions, variations, alterations, and modifications may be ascertained to one skilled in the art and it is intended that the present invention encompass all such changes, substitutions, variations, alterations, and modifications as falling within the spirit and scope of the appended claims. In order to assist the United States Patent and Trademark Office (USPTO) and additionally any readers of any patent issued on this application in interpreting the claims appended hereto, Applicant wishes to note that the Applicant: (a) does not intend any of the appended claims to invoke paragraph six (6) of 35 U.S.C. section 112 as it exists on the date of filing hereof unless the words “means for” are specifically used in the particular claims; and (b) does not intend by any statement in the specification to limit his invention in any way that is not otherwise reflected in the appended claims.

What is claimed is:

1. A device to be used in a cardiovascular environment, comprising:
an expandable element that is coupled to a rod and that includes a compressed state and an expanded state, wherein the expandable element is operable to be positioned within a wall of an organ while the expandable element is in the compressed state, and wherein the expandable element is further operable to be deployed once it is within the organ such that it is in the expanded state; and
a cutter element operable to make a circular incision at the wall of the organ, wherein the expandable element creates a resistive force when it is pulled against the wall while in the expanded state such that an interface is created for making the incision, and wherein the device includes a stop-grip mechanism that maintains the resistive force at the interface.

2. The device of claim 1, further comprising:
a stop and a grip element, wherein when the stop and the grip element are simultaneously squeezed to exert a pressure in order to produce the resistive force at the interface.

3. The device of claim 1, wherein the cutter element is actuated by rotating the cutter element along a threaded portion of the device.

4. The device of claim 1, further comprising:
a tube operable to hold the device for entry into a targeted location.

5. The device of claim 1, further comprising:
an obturator disposed on one end of the expandable element and operable to guide the expandable element into the wall of tissue.

6. The device of claim 5, wherein the obturator is removable.

7. The device of claim 1, further comprising:
a diaphragm and a valve that collectively serve as a base for the device; and
a port coupled to the device and operable to exhaust air and debris from a system in which the device is resident.

8. The device of claim 7, wherein the diaphragm, the port, and the valve are removable to allow a valve conduit to be provided over the rod and then subsequently inserted in the hole.

9. The device of claim 1, wherein the device is of a size sufficient to allow a sealed access to a cavity of the organ for placement, manipulation, or repair of a heart valve.

10. The device of claim 1, further comprising:
a handle operable to manipulate the expandable element into its compressed state and the expanded state.

11. The device of claim 1, wherein the cutter element includes a tissue retaining element.

12. A method to be performed in a cardiovascular environment, comprising:
positioning an expandable element that is coupled to a rod and that includes a compressed state and an expanded state within a wall of an organ while the expandable element is in the compressed state;
deploying the expandable element once it is within the organ such that it is in the expanded state; and
making an incision with a cutter element operable to make a circular incision at the wall of the organ, wherein the expandable element creates a resistive force when it is pulled against the wall while in the expanded state such that an interface is created for making the incision, and wherein the device includes a stop-grip mechanism that maintains the resistive force at the interface.

13. The method of claim 12, wherein the device is of a size sufficient to allow a sealed access to a cavity of the organ for placement, manipulation, or repair of a heart valve.

14. The method of claim 12, further comprising:
manipulating a handle coupled to the expandable element to control whether the expandable element is in the compressed state or the expanded state.

15. The method of claim 12, further comprising:
simultaneously squeezing a stop and a grip element in order to exert a pressure in order to produce the locking position that maintains the interface.

16. The method of claim 12, further comprising:
actuating the cutter element by advancing the cutter element along the rod of the device.

17. The method of claim 12, further comprising:
utilizing a port coupled to the device to exhaust air and debris from a system in which the device is resident; and
removing the port.

18. The method of claim 12, further comprising:
positioning a valve conduit at the hole;
manipulating the expandable element such that it is in the compressed state; and
removing the expandable element from the hole.

19. The method of claim 12, further comprising:
coating the expandable element with an elastomeric membrane in order to facilitate sealing of the interface.

20. The method of claim 12, wherein the device is of a size sufficient to allow a sealed access to a cavity of the organ for placement, manipulation, or repair of a heart valve.
